(19) United States
(12) Patent Application Publication
(10) Pub. No.: US 2022/0015986 A1
(43) Pub. Date: Jan. 20, 2022

(54) THREE-STEP PROCESS FOR MIMICKING PLASTIC SURGERY RESULTS

(71) Applicant: Hillary Hayman, Los Angeles, CA (US)

(72) Inventor: Hillary Hayman, Los Angeles, CA (US)

(21) Appl. No.: 16/930,760

(22) Filed: Jul. 16, 2020

(51) Int. Cl. A61H 15/02; A61N 5/06; A61H 15/00

(52) U.S. Cl. CPC A61H 15/02; A61N 5/0616; A61N 5/062; A61H 2201/1253; A61N 2005/0663; A61N 2005/0644; A61H 2015/0014; A61H 15/0092

(57) ABSTRACT

A three-step process performs, with a derma roller, a plurality of movements along an area of intended rejuvenation of a human user. Furthermore, the three-step process applies, subsequent to the derma roller performance, a chemical compound to one or more patches of skin at the area of intended rejuvenation. Finally, the three-step process emits, subsequent to the application of the chemical compound, light toward the area of intended rejuvenation. The three-step process may be customized to a user based on a variety of factors (e.g., area of intended rejuvenation, age, skin sensitivity, or the like).

### BACKGROUND

### 1. Field

This disclosure generally relates to skincare processes. More particularly, the disclosure relates to a process for mimicking plastic surgery results.

### 2. General Background

A plastic surgery procedure is typically performed in an office of a plastic surgeon, often at significant expense and inconvenience for those people wanting to improve the appearance of their skin. For instance, many people typically have to travel far distances to obtain the services of a qualified plastic surgeon to perform a plastic surgery procedure. Although some people may elect to have a plastic surgery procedure (e.g., facelift, jaw lift, eye wrinkle reduction, etc.) to rejuvenate the appearance of their skin, others require plastic surgery to diminish or eliminate the effects (e.g., scar tissue reduction, burn minimization, etc.) from injuries. As a result, plastic surgery procedures are typically accessible only to a limited group, which may exclude those people who need plastic surgery procedures the most.

Furthermore, even when people undergo a plastic surgery procedure, risks are associated therewith. In some instances, those undergoing plastic surgery procedures may end up with significant disfiguration. Although the skill and experience of the plastic surgeon may play a part, other factors, such as the way the skin heals for different people, may also lead to potential disfiguration. Ultimately, plastic surgery procedures often involve significant levels of invasiveness that may injure the skin of the person to whom the plastic surgery procedure is applied.

Yet, alternative, non-invasive approaches have had questionable, if any, efficacy. For example, one alternative approach utilizes micro current technology to stimulate adenosine triphosphate (“ATP”) production in an attempt to create collagen and elastin to provide a lifted appearance of the skin. However, this approach appears to have results that do not mirror plastic surgery results. Another approach may utilize radio frequency (“RF”) emissions in conjunction with micro current emissions, but this approach may lead to results that worsen the skin, such as excessive redness. Yet another approach purports to oxygenate the skin to improve blood flow, but has led to health concerns, such as a racing heart, chest pressure, and light headedness.

Accordingly, current skincare configurations suffer from procedures that are either too invasive (i.e., plastic surgery procedures that are costly, inconvenient, and vulnerable to possible disfiguration) or too non-invasive (i.e., devices that rely solely on technologies that emit electricity, radio waves, air, or the like above the surface of the skin of a person).

### SUMMARY

In one embodiment, a three-step process performs, with a derma roller, a plurality of movements along an area of intended rejuvenation of a human user. Furthermore, the three-step process applies, subsequent to the derma roller performance, a chemical compound to one or more patches of skin at the area of intended rejuvenation. Finally, the three-step process emits, subsequent to the application of the chemical compound, light toward the area of intended rejuvenation. The three-step process may be customized to a user based on a variety of factors (e.g., area of intended rejuvenation, age, skin sensitivity, or the like).

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1A illustrates the apparatuses associated with the first two steps of the three-step process.

FIG. 11B illustrates the apparatus associated with the third step of the three-step process.

FIG. 2A illustrates the user moving the shaft of the derma roller in a vertical motion along her face.

FIG. 2B illustrates the user moving the shaft of the derma roller in a diagonal motion along her face.

FIG. 2C illustrates the user applying the chemical compound to one or more patches of a predetermined patch size, such as four inches by four inches.

FIG. 2D illustrates the user utilizing the red light therapy emission device.

FIG. 3A illustrates the user utilizing the derma roller to perform movements on various portions of the stomach.

FIG. 3B illustrates the user applying the chemical compound to the intended area of rejuvenation on the stomach.

FIG. 3C illustrates the user emitting red light, via the red light therapy device, toward the intended area of rejuvenation on the stomach.

FIG. 4 illustrates the three-step process.

FIG. 5A illustrates the skin during movement of the derma roller head as the derma roller needles puncture an epidermis layer of the skin to create a plurality of channels via the epidermis layer, and potentially the dermis layer.

FIG. 5B illustrates the chemical compound filling the various channels created by the derma roller.

FIG. 5C illustrates various red light emissions being emitted toward the chemical compound situated in the channels to enhance collagen production to mimic plastic surgery results.

### DETAILED DESCRIPTION

A three-step process provides users with skincare rejuvenation that mimics plastic surgery results, without users having to undergo conventional plastic surgery procedures within a plastic surgeon's office. By way of contrast with conventional processes, the three-step process may be performed from the convenience of one's own home, or other place of comfort.

In particular, the three-step process has been discovered to yield results that were not readily predictable; such results mimic the effects of various plastic surgery procedures (e.g., facelifts, jaw lifts, neck lifts, forehead lifts, lower eyelid blepharoplasty, and the like) without a user having to undergo a plastic surgery procedure at the office of a medical practitioner. In essence, the three-step process includes application of various invasive and non-invasive devices/products according to particular parameters to mimic plastic surgery results. Firstly, the three-step process includes usage of a derma roller in an area of intended rejuvenation. Particular needle lengths and movements of the derma roller have been discovered to maximize skin rejuvenation. Secondly, a chemical compound containing vitamin A₁ (e.g., Retinol) is applied to the area of intended skin rejuvenation via the channels in the skin created by the derma roller. Accordingly, the chemical compound is able to reach the dermis layer of the skin, directly underneath the epidermis, much faster than would be required through prolonged exposure to the chemical compound; as a result, collagen production is increased in an optimal manner to rejuvenate (i.e., tone, tighten, and/or lift) the skin. Finally, a red light therapy device is utilized to emit red light (i.e., light having a wavelength of six hundred ten nanometers to seven hundred nanometers) toward the area of intended rejuvenation, which further increases collagen production.

Accordingly, the positioning of the chemical compound within the channels created by derma roller, combined with the sequential step of red light emission toward the area of intended rejuvenation, has been discovered to have an enhanced collagen production effect that mimics plastic surgery results.

Furthermore, the same three-step process may be utilized in a universal manner with respect to various portions of the face to mimic plastic surgery results corresponding to different plastic surgery procedures (e.g., face lift, jaw lift, etc.), but with the same process. In other words, one process may be utilized in different zones of the face of a user to mimic plastic surgery results that would typically require different plastic surgery procedures, associated with potentially different visits to a plastic surgeon's office.

Furthermore, in one embodiment, the three-step process may be utilized to simultaneously mimic the results from multiple plastic surgery procedures during one application. For example, the user may apply the derma roller to both the skin around the eyes and the skin around the jaw during one application, followed by application of retinol to the skin around the eyes and the skin around the jaw, and followed by red light therapy emission to the skin around the eyes and the skin around the jaw. Accordingly, the three-step process allows a user to simultaneously mimic plastic surgery results corresponding to multiple plastic surgery procedures at the same time, and by using the same process, without necessitating any variation thereto.

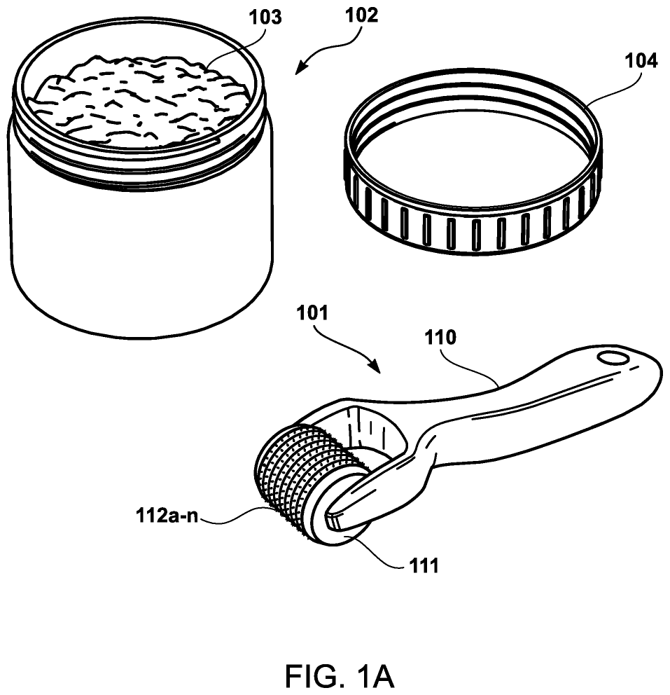

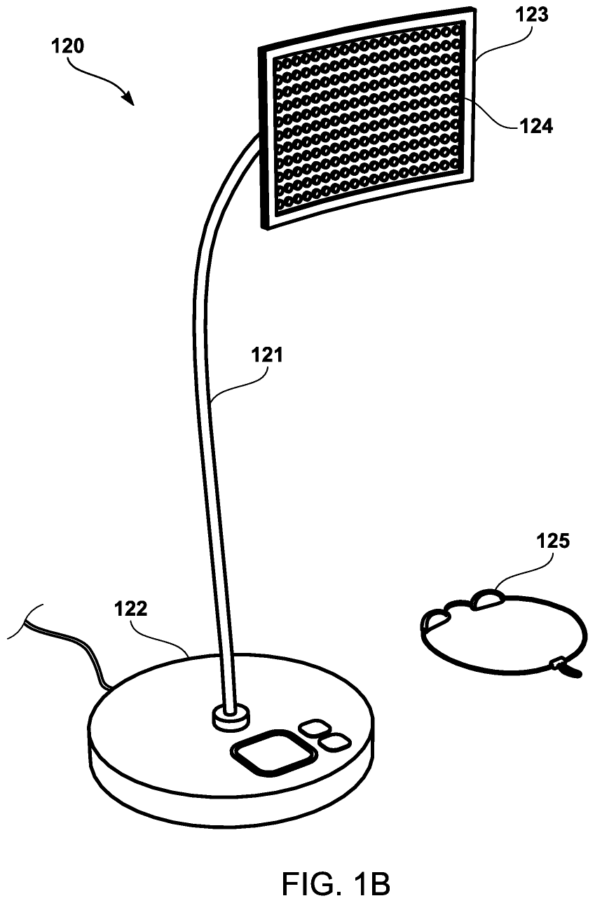

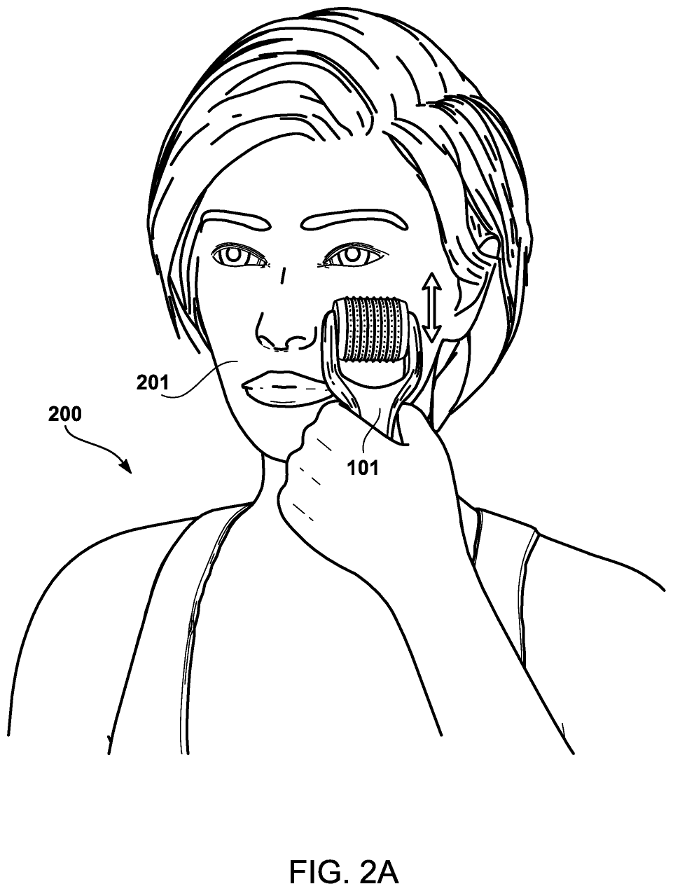

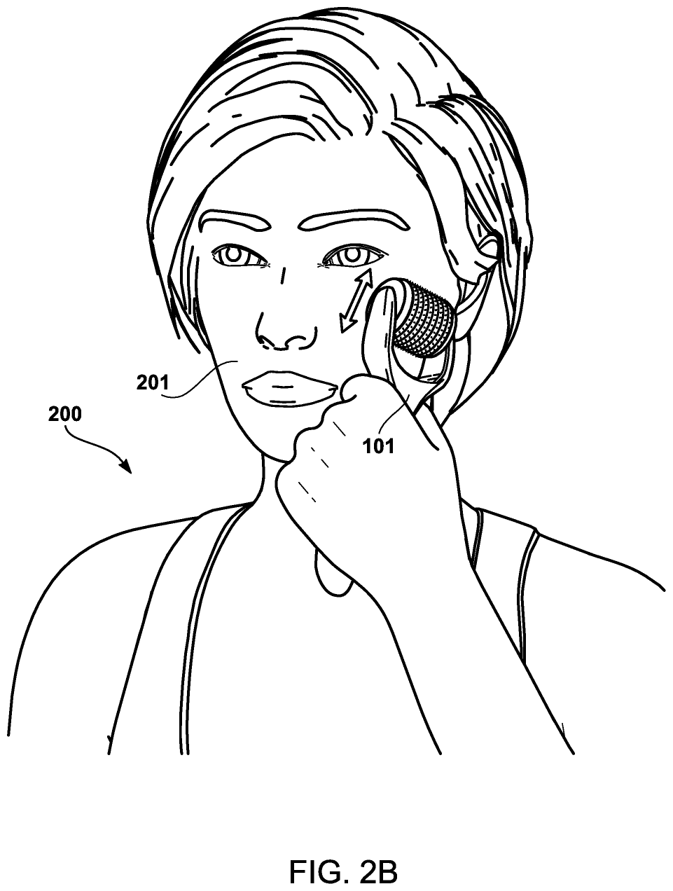

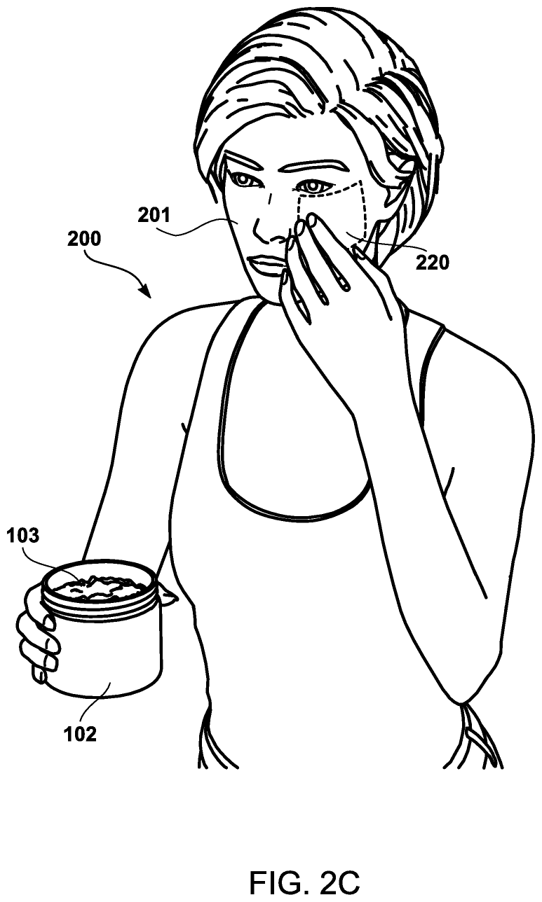

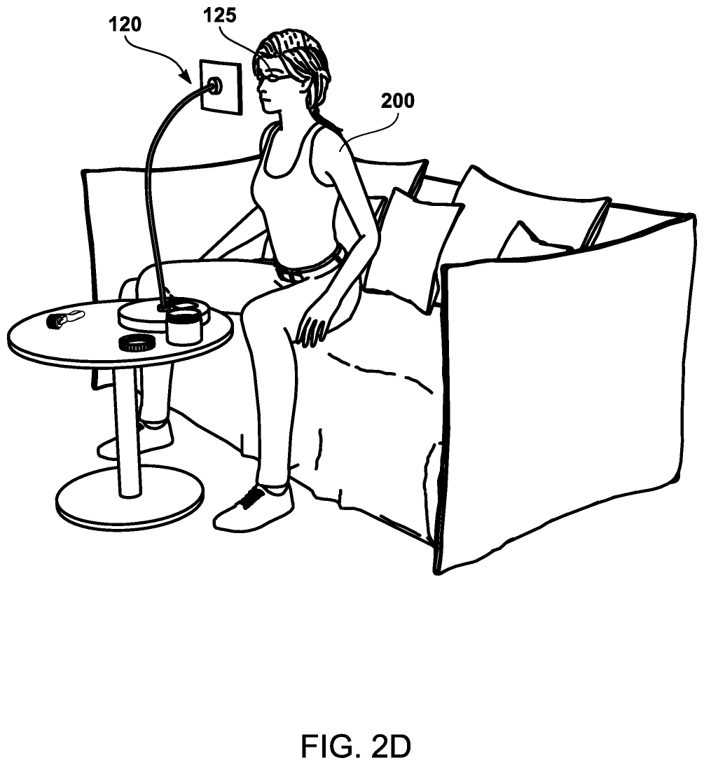

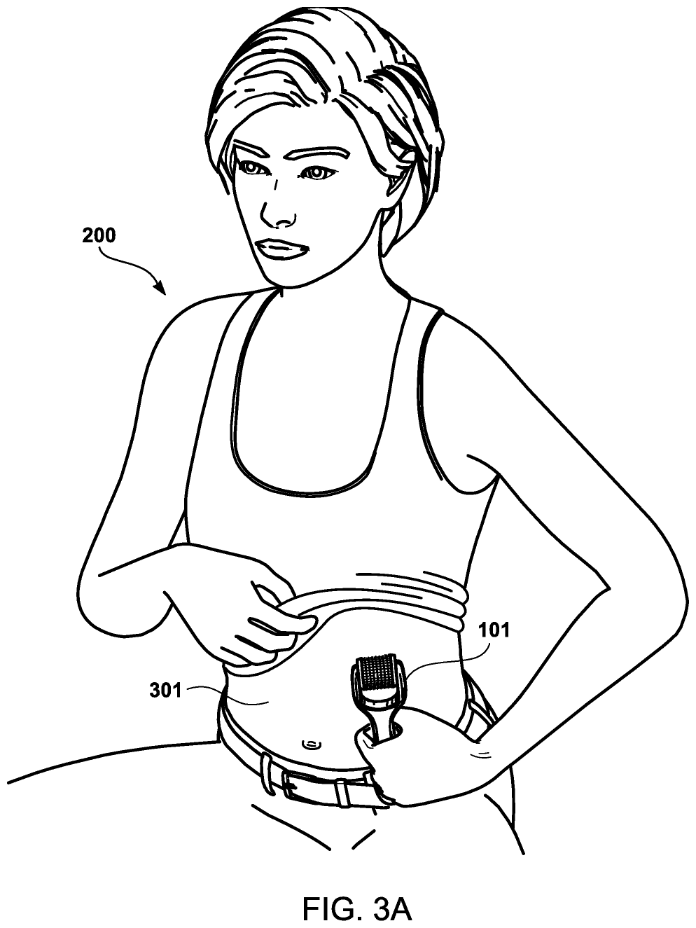

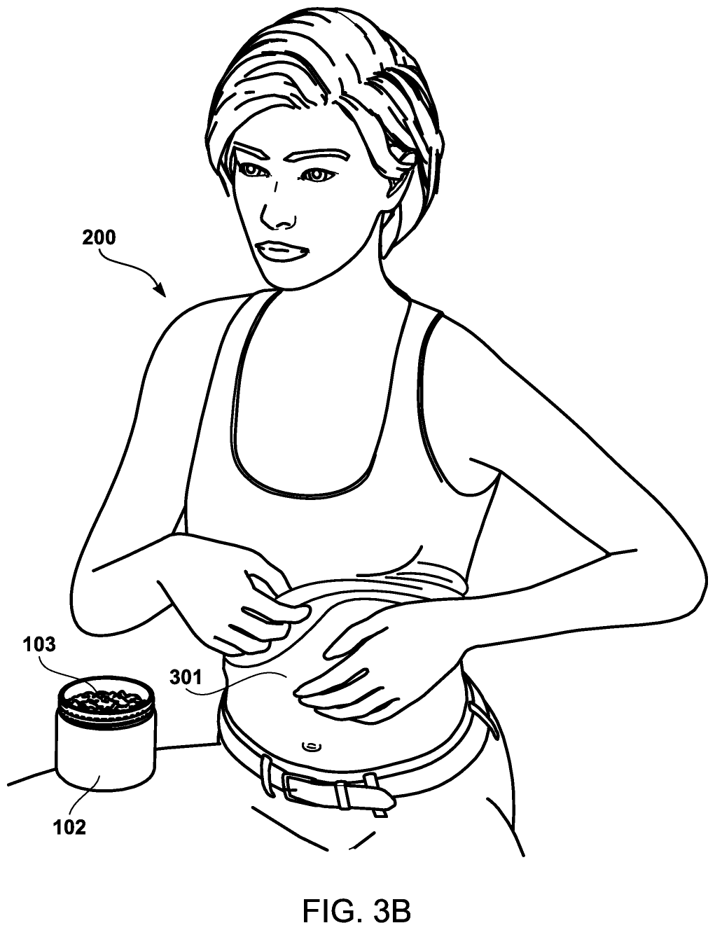

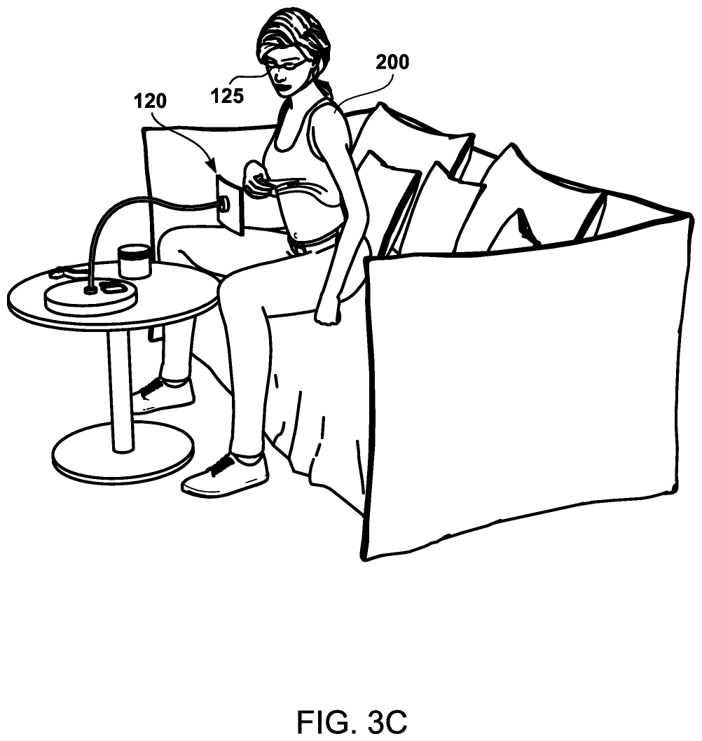

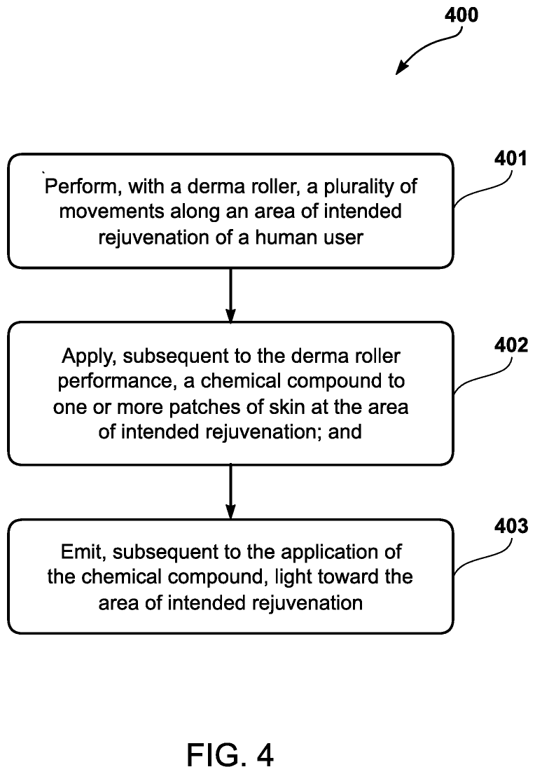

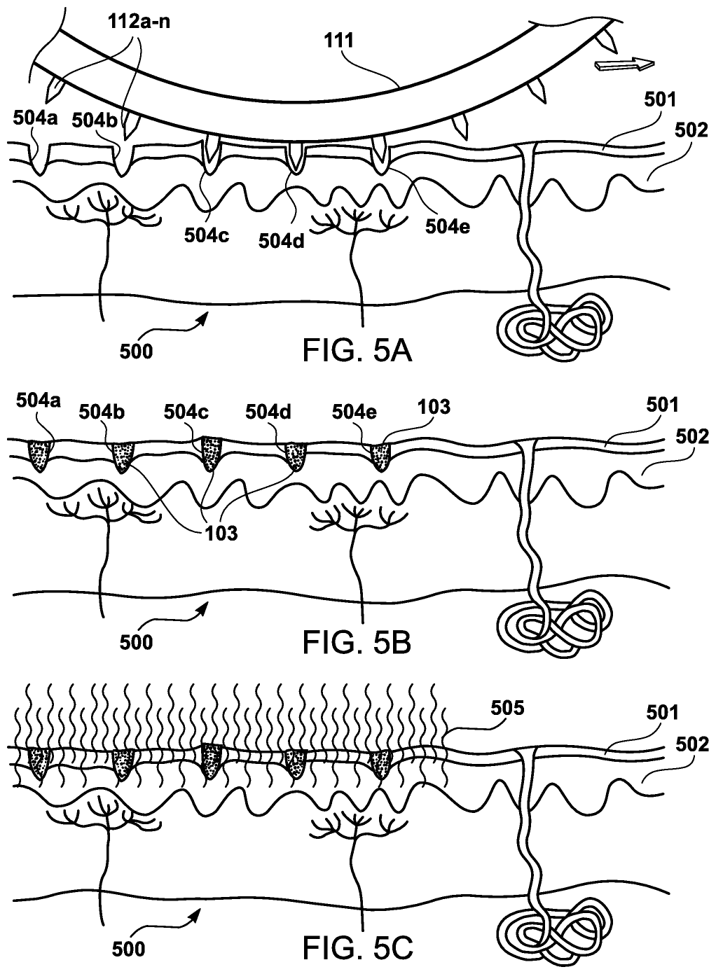

FIGS. 1A and 1B illustrate examples of the various apparatuses utilized as part of the three-step process. In particular, FIG. 1A illustrates the apparatuses utilized during the first two steps of the three-step process. At step one of the three-step process, a derma roller 101 is utilized to puncture a plurality of channels within the skin of a user. The derma roller 101 has a shaft 110 that is operably connected to a derma roller head 111. A plurality of needles 112a-n are situated around the circumference of the derma roller head 111 to allow for movement of at least a portion of the plurality of needles 112a-n along the skin of the user based upon movement of the shaft 110.

Of particular note, in one embodiment, the three-step process utilizes needles that are 0.5 mm in length, since this particular needle length has been discovered to be an optimal minimum needle length for skincare rejuvenation—especially for beginning users of the three-step process. As users progressively utilize the three-step process over an extended period of time (e.g., weeks, months, and/or years), users may utilize a needle length ranging between 0.5 mm and 2.0 mm at an intermediate level, and ultimately 2.0 mm for maximum skincare rejuvenation at an advanced level. (The foregoing parameters are provided only as examples of particular needle lengths. Slight variations (e.g., one to thirty percent differences in needle lengths) of these parameters potentially may be utilized to obtain similar results.) In an alternative embodiment, a micro needling apparatus may be utilized instead of the derma roller 101 to puncture the skin of the user via direct vertical movements. Various other apparatuses having one or more needles attached thereto may be utilized instead.

Furthermore, at step two of the three-step process, a chemical compound 103 may be stored in a container 102 for use after the user performs movements with the derma roller 101. In one embodiment, the chemical compound 103 is applied to patches of skin substantially equaling (i.e., without exceeding a thirty to forty percent additional size threshold) four inch by four inches in size. Application of the chemical compound 103 to patches of these particular sizes of skin has been discovered to be a factor in the three-step process of skincare rejuvenation. In essence, the application of the chemical compound 103 is utilized for wrinkle repair and smoothing out the texture of the skin. The use of the derma roller 101 in the first step allows for increased efficacy of the chemical compound 103 because it is better absorbed via the channels created by the derma roller 101.

Finally, at step three of the three-step process, as illustrated by FIG. 2B, a red light therapy machine 120 is utilized to emit red light toward the area of the skin of intended rejuvenation. Although illustrated with a base 122 and a stand 121 to allow for hands-free usage, in an alternative embodiment, the red light therapy machine 120 may be a handheld device. In one embodiment, the three-step process involves exposure from one or more light emitting diodes (“LEDs”) positioned on a panel 124 of the red light therapy machine 120 for a time period substantially equaling (i.e., within an approximate ten percent threshold) a minimum of ten minutes; this time duration has been discovered to be the time duration that maximizes skincare rejuvenation. The use of the red light therapy machine 120 has been discovered to increase the efficacy of the derma roller 101 and the chemical compound 103 by an approximate factor of four. Optionally, a pair of light emission googles 125 may be utilized by the user to protect his or her eyes during light emission.

FIGS. 2A-2D illustrate a user 200 utilizing the three-step process to mimic results from a facelift. In particular, FIG. 2A illustrates the user 200 moving the shaft 110 of the derma roller 101 in a vertical motion along her face 201. (The user 200 is illustrated as a female only as an example. Accordingly, the three-step process also may be utilized by a male.) In one embodiment, a predetermined maximum quantity of four vertical motions, at medium pressure (e.g., pressure in substantially in the range of ten to fifty pounds per square inch) over the area of intended skin rejuvenation, has been discovered to provide optimal skincare rejuvenation results. (The foregoing example range is provided only as an example of a pressure that is not too light to be ineffective, but also not too strong (e.g., one hundred pounds per square inch) to beak the skin without a needle.) In particular, more than four vertical motions has been determined to lead to less optimal results, potentially leading to too much exertion on the skin. Furthermore, FIG. 2B illustrates the user 200 moving the shaft 110 of the derma roller 101 in a diagonal motion along her face 201. In one embodiment, a predetermined maximum quantity of four diagonal motions, at medium pressure, over the area of intended skin rejuvenation has been discovered to provide optimal skincare rejuvenation results. Moreover, in one embodiment, the movement of the derma roller 101 is specific to vertical and diagonal movements, to the exclusion of horizontal movements, which has been discovered to potentially limit the effectiveness of the vertical and diagonal movements—potentially moving the skin in an opposite direction to the lifting effect of the vertical and diagonal movements. In another embodiment, horizontal movements may be utilized in conjunction with the vertical and diagonal movements. In yet another embodiment, vertical movements or diagonal movements may be utilized to the exclusion of the other. Finally, in another embodiment, the predetermined maximum quantity of motions may differ from four motions. For example, based on a variety of factors (e.g., age, skin type, etc.), a different quantity of motions may be utilized to maximize skincare rejuvenation.

FIG. 2C illustrates the user 200 applying the chemical compound 103 to one or more patches 220 of skin of a predetermined patch size, such as four inches by four inches. As an example, the user 200 may apply a dime-sized portion of retinol to the predetermined patch size 221 portion of her skin.

Finally, after applying the chemical compound 103, the user 200 may utilize the red light therapy emission device 120, as illustrated in FIG. 2D. The user 200 may wear protective eyewear 125 during the red light therapy emission to protect her eyes. In one embodiment, the optimal minimal exposure time is substantially at least ten minutes.

Although, the three-step process is illustrated in FIGS. 2A-2D as being implemented to obtain skincare rejuvenation results that mimic a facelift, the same process may be utilized to mimic other skincare rejuvenation results associated with the face. As a result, the user 200 may utilize one universal process for skincare rejuvenation on all parts of the face having skin for possible skincare rejuvenation.

Furthermore, the three-step process may be adapted for use on the body of the user 200. FIGS. 3A-3C illustrate application of the three-step process to the stomach 301 of the user 200 to remove stretch marks. In particular, the user 200 may utilize the derma roller 101 to perform movements on various portions of the stomach 301, as illustrated by FIG. 3A. Whereas a derma roller length of 0.5 mm is appropriate for the face, a derma roller length of one and one half mm may be utilized for the stomach, given that the skin of the stomach 301 is generally thicker than the skin of the face. Furthermore, as illustrated in FIG. 3B, the user 200 may apply the chemical compound 301 to the intended area of rejuvenation on the stomach 301, via one or more patches 220. Additional chemical compounds, such as sun protection factor (“spf”) formulated lotion, may be applied to the stomach 301 in conjunction with the chemical compound 103. Optionally, caffeinated lotion therapy may be utilized for an immediate effect. Finally, as illustrated in FIG. 3C, the user 200 may emit red light, via the red light therapy device 120, toward the intended area of rejuvenation on the stomach 301. In an alternative embodiment, additional, or different, light emission devices may be utilized for emitting light toward the stomach 301. For example, a blue light therapy device, which emits blue light at a wavelength between four hundred five nanometers to four hundred twenty nanometers, may be utilized to emit additional or different light than the red light therapy device 120. Alternatively, a universal light emission device, which emits both red light and blue light in response to different commands, may be utilized instead. (The stomach 301 is illustrative of only one example of how the three-step process may be utilized on a body part other than the face 201. Accordingly, the three-step process may be utilized on a variety of other body parts.)

As another example, the three-step process may be utilized to reduce pores of the user 200, on the face 201 or the body by utilizing caffeine in addition, or in the alternative, to the retinol, as well as assorted light therapy. As yet another example, the three-step process may be utilized to remove rosacea by utilizing a moisturizing agent as the chemical compound 103, and blue light therapy instead of red light therapy, to reduce, minimize, or dissolve surface level capillaries. Additionally, another example is the three-step process being utilized to remove melasma (e.g., dark spots on the face and body) by utilizing a spot removing agent as the chemical compound 103, and assorted light therapy.

In addition to the chemical compound 103 described with respect to FIG. 1A, various other chemical compounds may be utilized to alleviate dryness, to disinfect the affected area, and numb the affected area. For example, a barrier cream and/or moisturizer cream may be utilized to help with dryness. Furthermore, a disinfecting agent may be utilized prior to and/or after use of the derma roller 101 to disinfect it. Also, a disinfecting agent that is alcohol free may be applied to the face before applying numbing cream.

FIG. 4 illustrates the three-step process 400. At a process block 401, the three-step process 400 performs, with the derma roller 101, a plurality of movements along an area of intended rejuvenation of a human user. Furthermore, at a process block 402, the three-step process 400 applies, subsequent to the derma roller performance, a chemical compound 103 to one or more patches of skin at the area of intended rejuvenation. Finally, at a process block 403, the three-step process emits, subsequent to the application of the chemical compound, light toward the area of intended rejuvenation. The three-step process may be customized to a user 200 based on a variety of factors (e.g., area of intended rejuvenation, age, skin sensitivity, or the like).

FIGS. 5A-5C illustrate various areas of the skin 500 of the user 200 during the three-step process. In particular, FIG. 5A illustrates the skin 500 during movement of the derma roller head 111 as the derma roller needles 112a-n puncture an epidermis layer 501 of the skin to create a plurality of channels 504a-e (more channels may be punctured than those illustrated) via the epidermis layer 501, and potentially the dermis layer 502. Furthermore, FIG. 5B illustrates the chemical compound 103 filling the various channels 504a-e created by the derma roller 101. Finally, FIG. 5C illustrates various red light emissions 505 being emitted toward the chemical compound 103 situated in the channels 504a-504e to enhance collagen production to mimic plastic surgery results.

It is understood that the processes, systems, and apparatuses described herein may also be applied in other types of processes, systems, and apparatuses. Those skilled in the art will appreciate that the various adaptations and modifications of the embodiments of the processes, systems, and apparatuses described herein may be configured without departing from the scope and spirit of the present processes and systems. Therefore, it is to be understood that, within the scope of the appended claims, the present processes, systems, and apparatuses may be practiced other than as specifically described herein.

1: A process comprising:
performing, with a derma roller, a predetermined maximum number of movements in each of one or more predetermined directions along an area of intended rejuvenation of a face of a human user, wherein the one or more predetermined directions are selected from a diagonal motion and a vertical motion to impart a lifting effect on a skin of the user, wherein the one or more predetermined directions exclude a horizontal motion, wherein the predetermined maximum number of movements equals four, wherein the derma roller comprises a head with a plurality of needles, wherein the plurality of needles each have a length of five tenths millimeters situated thereon for beginning users, wherein the plurality of needles each have a length between five tenths millimeters and two millimeters situated thereon for intermediate users, wherein the plurality of needles each have a length of two millimeters situated thereon for advanced users, and wherein the plurality of needles of the derma roller puncture a plurality of channels within the skin of the user;
applying, subsequent to the derma roller performance, a chemical compound to one or more patches of skin at the area of intended rejuvenation, the one or more patches of skin each having a predetermined patch size, the chemical compound comprising vitamin A₁, the predetermined patch size equaling four inches in length by four inches in width; and
emitting, subsequent to the application of the chemical compound, red light toward the area of intended rejuvenation for a minimum predetermined period of time equaling ten minutes within a ten percent threshold, the red light having a wavelength in the range of six hundred ten nanometers to seven hundred nanometers.

2: The process of claim 1, wherein the predetermined patch size is within a forty percent additional size threshold.

3: The process of claim 1, wherein the chemical compound is absorbed via the plurality of channels within the skin of the user.

4: The process of claim 3, wherein the chemical compound is configured to repair wrinkles and smooth out a texture of the skin of the user.

5: The process of claim 1, wherein the performance with the derma roller occurs at a pressure in the range of ten pounds per square inch to fifty pounds per square inch, and wherein the emitted red light is configured to act on the chemical compound absorbed in the plurality of channels to enhance collagen production by a factor of four as compared to use of the derma roller and chemical compound alone.

6: The process of claim 1, wherein the area of intended rejuvenation is selected from the group consisting of: skin around one or more eyes of the user, skin around one or more cheeks of the user, skin around a forehead of the user, and skin around a jaw of the user.

7: A process comprising:
performing, with a derma roller, a plurality of movements along an area of intended rejuvenation of a human user, wherein the derma roller punctures a plurality of channels within a skin of a user;
applying, subsequent to the derma roller performance, a chemical compound to one or more patches of skin at the area of intended rejuvenation, wherein the chemical compound is configured to repair wrinkles and smooth out a texture of the skin of the user; and
emitting, subsequent to the application of the chemical compound, light toward the area of intended rejuvenation, wherein the emitted red light is configured to act on the chemical compound absorbed in the plurality of channels to enhance collagen production by a factor of four as compared to use of the derma roller and chemical compound alone.

8: The process of claim 7, wherein the area of intended rejuvenation is at least a portion of a face of the human user.

9: The process of claim 8, wherein the derma roller has a head with a plurality of needles each having a length in a range of five tenths millimeters to two millimeters situated thereon.

10: The process of claim 9, wherein the chemical compound comprises vitamin A₁.

11: The process of claim 10, wherein the chemical compound is applied to one or more patches of skin at the area of intended rejuvenation, the one or more patches of skin each having a predetermined patch size equaling four inches in length by four inches in width.

12: The process of claim 9, wherein the light is red light having a wavelength in the range of six hundred ten nanometers to seven hundred nanometers.

13: The process of claim 7, wherein the area of intended rejuvenation is at least a portion of a stomach of the human user.

14: The process of claim 13, wherein the derma roller has a head with a plurality of needles each having a length in a range of one and one half millimeters to two millimeters situated thereon.

15: The process of claim 14, wherein the chemical compound comprises vitamin A₁.

16: The process of claim 14, wherein the chemical compound comprises caffeinated lotion.

17: The process of claim 14, wherein the chemical compound is applied to one or more patches of skin at the area of intended rejuvenation, the one or more patches of skin each having a predetermined patch size equaling four inches in length by four inches in width.

18: The process of claim 14, wherein the light comprises red light and blue light, the red light having a wavelength in the range of six hundred ten nanometers to seven hundred nanometers, the blue light having a wavelength in the range of four hundred five nanometers to four hundred twenty nanometers.

19: The process of claim 14, wherein the plurality of movements are performed in one or more predetermined directions comprising a vertical direction and a diagonal direction to an exclusion of any horizontal direction.

20: The process of claim 19, wherein the performance with the derma roller occurs at a pressure in the range of ten pounds per square inch to fifty pounds per square inch.
